(19) United States
(12) Patent Application Publication
(10) Pub. No.: US 2012/0258168 A1
(43) Pub. Date: Oct. 11, 2012

(54) METHODS AND COMPOSITIONS TO PROMOTE OCULAR HEALTH

(75) Inventor: Carlos A. Montesinos, Katy, TX (US)

(73) Assignee: AMERISCIENCES, LP, Houston, TX (US)

(21) Appl. No.: 13/442,302

(22) Filed: Apr. 9, 2012

Related U.S. Application Data
(60) Provisional application No. 61/472,779, filed on Apr. 7, 2011.

(30) Foreign Application Priority Data
Apr. 27, 2011 (CA) 2738357

(51) Int. Cl. A61K 33/30; A61P 27/02; A61K 9/48; A61K 33/34; A61K 9/28

(52) U.S. Cl. 424/455; 424/638; 424/641; 424/474

(57) ABSTRACT

Embodiments include a composition to promote ocular health and a method of treatment for a subject exposed to a source of oxidative or visual stress to the eye or having a degradation of the eye. The composition may include amounts of vitamin A, which includes beta-carotene; vitamin C; vitamin D; vitamin E; zinc; copper; selenium; non-vitamin A carotenoids, which include lutein and zeaxanthin; omega-3 fatty acids, which include eicosapentaenoic acid and docosahexaenoic acid; taurine; alpha lipoic acid; pine bark extract; astaxanthin; and Piper spp. extract. The method includes the step of administering to the subject a daily dose of a composition to promote ocular health.

### CROSS-REFERENCE TO RELATED PATENT APPLICATIONS

This application claims priority from U.S. Provisional Application No. 61/472,779, filed Apr. 7, 2011, and from Canadian Patent Application No. 2,738,357, filed Apr. 27, 2011. For purposes of United States patent practice, this application incorporates the contents of these applications by reference in their entirety.

### BACKGROUND OF THE INVENTION

1. Field of the Invention

The field of invention relates to compositions and methods useful for to promote ocular health of a subject. More specifically, the field of invention relates to compositions and methods for ameliorating oxidative and visual stresses and degradation of the eye, including age-related macular degeneration (AMD).

2. Description of the Related Art

The eyes play an important role in mobility, function, and enjoyment of life. For this reason, it is important to maintain good ocular health. The term “ocular” refers to the eye and its organ system. Unfortunately, ocular health declines naturally with age. This natural decline can be attributable to many things, including exposure to ultraviolet light from the sun, wind, dust, chlorine and other chemical fumes and liquids, automobile exhaust fumes, and physical injury.

### SUMMARY OF THE INVENTION

The invention includes a composition to promote ocular health. The composition include amounts of vitamin A, which includes beta-carotene; vitamin C; vitamin D; vitamin E; zinc; copper; selenium; non-vitamin A carotenoids, which include lutein and zeaxanthin; omega-3 fatty acids, which include eicosapentaenoic acid and docosahexaenoic acid; taurine; alpha lipoic acid; pine bark extract; astaxanthin; and Piper spp. extract. Embodiments of the composition optionally exclude beta-carotene. Embodiments of the composition optionally exclude vitamin E. Embodiments of the composition optionally exclude copper.

The invention includes a method of treatment for a subject exposed to a source of oxidative or visual stress to the eye or having a degradation of the eye, including age-related macular degeneration (AMD). The method includes the step of administering to the subject a daily dose of the compositions to promote ocular health. The administration is performed such that the effects induced by the oxidative or visual stress source or the degradation of the eye are ameliorated. Embodiments of the method include administration of the daily dose of the composition proportionally during a 24-hour period.

Embodiments of the method include a step of diagnosing the subject with the degradation of the eye. Embodiments include diagnosing the subject with the degradation of the eye due to age-related macular degeneration, diabetes or hyperglycemia.

### DETAILED DESCRIPTION OF THE PREFERRED EMBODIMENTS

The Specification, which includes the Summary of Invention, Brief Description of the Drawings and the Detailed Description of the Preferred Embodiments, and the appended Claims refer to particular features (including method steps) of the invention. Those of skill in the art understand that the invention includes all possible combinations and uses of particular features described in the Specification, including all of those features specifically described. For example, in describing a feature as part of an embodiment or an aspect of the invention, one of ordinary skill in the art understands that the described feature can and is used, to the extent possible, in combination with or in context of other features described as part of other embodiments and aspects of the invention.

Those of skill in the art understand that the invention is not limited to or by the description of embodiments as given in the Specification. Those of skill in the art also understand that the terminology used for describing particular embodiments does not limit the scope or breadth of the invention.

### Problem

Many of the compositions known heretofore have been focused solely on providing treatment for age-related visual decline. Many subjects, however, also suffer from poor ocular health due to other illnesses, including dry eye, visual acuity, diabetes, hyperglycemia, and increased levels of visual stress due to long amounts of exposure to visual display terminals, including computer monitors, smart phones, laptops, and tablet personal computers.

Therefore, it would be advantageous to provide methods and compositions to promote ocular health that did not suffer from these shortcomings.

### Solution

Compositions having low levels of certain vitamins, trace elements, antioxidants, and fatty acids promote ocular health and provide the benefits of improved health and well-being for a subject, which includes mammals, which especially includes humans (homo sapiens). Some compositions that promote ocular health specifically exclude beta-carotene and Vitamin E. Some compositions include lutein, zeaxanthin, alpha lipoic acid, vitamin D and astaxanthin, and do not include beta-carotene and pro-vitamin A (PVA) carotenoids. Some compositions provide support of polyphenolics.

Compositions that promote ocular health can be effective for subjects exposed to visually stressful situations, including working with visual display terminals for extended periods. The composition can provide vasoprotective effects, anti-inflammatory properties, and improvement in capillary function of the eye. The compositions can treat and improve the health of subjects with age-related macular degeneration (AMD). The compositions can promote ocular health by use as a multi-factorial nutritional adjuvant for subjects seeking to protect and strengthen their eyes, vision, lacrimal function. The compositions can also support daily dietary needs, and particularly if the subject is at risk for increased oxidative stress in their retina (i.e. hyperglycemics and diabetics).

Compositions that promote ocular health can include certain omega-3 type fatty acids. Fatty acids, specifically fatty acids obtained from fish oil, have been found to have a number of beneficial health effects. Oils from fish can contain eicosapentaenoic acid (EPA) and docosahexaenoic acid (DHA), which are omega-3 fatty acids. These omega-3 fatty keep blood triglycerides in check and may inhibit the progression of atherosclerosis. Although not intending to be bound by theory, it is believed that EPA and DHA have anti-inflammatory activity and are sometimes used as dietary supplements with inflammatory conditions, such as Crohn's disease and rheumatoid arthritis. It is also believed that the omega-3 fish oil fatty acids may balance other fatty acids. When fatty acids are out of balance in the body, the body can release chemicals that promote inflammation. Prostaglandins require omega-3 fatty acids. Prostaglandins are hormone-like substances that regulate dilation of blood vessels, inflammatory responses, and other critical body processes.

It is further believed that DHA and EPA are also essential for nerve and eye functions. DHA comprises about 60 percent of the outer rod segments of photoreceptor cells that are used to see with by humans. DHA is the substantial component of fat in brain tissue. It is believed that fish oil omega-3 fatty acids, and specifically DHA and EPA, are useful in wet macular degeneration since these fatty acids help heal and support blood vessel walls. Studies show that eating fish several times a month may reduce the risk of developing AMD.

Although not intending to be bound by theory, it is believed that omega-3 fatty acids may slow the progression of vision loss and reverse the signs of dry eye syndrome. It is also believed that there is a relationship between essential fatty acid (EFA) supplementation and improvement in dry eyes and dry eye symptoms. No more than 5,000 mgs of omega-3 fatty acids in a nutritional supplement with any other ingredients will perform incremental vital function improvement in terms protecting against loss of visual acuity due to various eye diseases, including AMD.

### Compositions to Promote Ocular Health

Compositions to promote ocular health are a mixture that can include vitamins A, some of which can be beta-carotene, C, D, E; zinc; copper; selenium; lutein; zeaxanthin; eicosapentaenoic acid; docosahexaenoic acid; taurine; alpha lipoic acid; pine bark extract; astaxanthin; and Piper spp. extract. Some embodiment compositions comprise vitamins A, C, and D; zinc; selenium; lutein; zeaxanthin; eicosapentaenoic acid; docosahexaenoic acid; taurine; alpha lipoic acid; pine bark extract; astaxanthin; and Piper spp. extract.

Units of measure for Tables 1-2 include “IU”, which represents “International Units”, an understood metric in the art for measuring the active amount of particular species, especially vitamins (e.g., Vitamins A, D, and E). Milligrams (“mg”) are 1×10⁻³ grams. Micrograms (“μg”) are 1×10⁻⁶ grams.

Table 1 shows the composition daily dose range of components for useful compositions to promote ocular health. Table 2 shows the daily dose of an embodiment composition to promote ocular health.

TABLE 1
Composition daily dose range of components for
useful compositions to promote ocular health.

| | | Units of |
|---|---|---|
| Component | Daily Dose Range | Measure |
| Vitamin A (pre-formed) | 2,500-50,000 | IU |
| Beta-Carotene (pro-vitamin A) | 0-50,000 | IU |
| Vitamin C | 60-500 | mg |
| Vitamin D | 400-2,000 | IU |
| Vitamin E (natural or synthetic) | 0-400 | IU |
| Zinc | 15-80 | mg |
| Copper | 0-2 (1-2 if zinc is | mg |
| | above 30 mg) | |
| Selenium | 35-200 | μg |
| Non-Vitamin A Carotenoid - Lutein | 5-50 | mg |
| Non-Vitamin A Carotenoid - Zeaxanthin | 0.25-12 | mg |
| Total Non-Vitamin A Carotenoids | 5-62 | mg |
| Omega-3 Fatty Acid - Eicosapentaenoic | 0-5,000 | mg |
| acid | | |
| Omega-3 Fatty Acid - Docosahexaenoic | 0-3,000 | mg |
| acid | | |
| Total Omega-3 Fatty Acids | 0-5,000 | mg |
| Taurine | 100-1,000 | mg |
| Alpha Lipoic Acid | 0-1,000 | mg |
| Pine Bark Extract | 0-500 | mg |
| Astaxanthin | 0-5 | mg |
| Piper spp. Extract | 0-5 | mg |

In some embodiments useful for promoting ocular health, the amount of total non-Vitamin A carotenoids is from about 0 to about 12 mg for the daily dose.

The amount of copper in the composition to promote ocular health depends on the amount of zinc present in the composition. In embodiment compositions where the amount of zinc is less than 30 mg, copper is not present in any amount. In embodiment compositions where the amount of zinc is equal to or greater than 30 mg, copper can be present in an amount in a range of from about 1 to about 2 mg.

TABLE 2
The daily dose of components for an embodiment
composition to promote ocular health.

| | | Units of |
|---|---|---|
| Component | Daily Dose | Measure |
| Vitamin A (pre-formed) | 2,500 | IU |
| Beta-Carotene (pro-vitamin A) | 0 | IU |
| Vitamin C | 250 | mg |
| Vitamin D | 800 | IU |
| Vitamin E (natural or synthetic) | 0 | IU |
| Zinc | 30 | mg |
| Copper | 0 | mg |
| Selenium | 70 | μg |
| Non-Vitamin A Carotenoid - Lutein | 10 | mg |
| Non-Vitamin A Carotenoid - Zeaxanthin | 2 | mg |
| Total Non-Vitamin A Carotenoids | 12 | mg |
| Omega-3 Fatty Acid - Eicosapentaenoic | 300 | mg |
| acid | | |
| Omega-3 Fatty Acid - Docosahexaenoic | 200 | mg |
| acid | | |
| Total Omega-3 Fatty Acids | 500 | mg |
| Taurine | 500 | mg |
| Alpha Lipoic Acid | 100 | mg |
| Pine Bark Extract | 10 | mg |
| Astaxanthin | 1 | mg |
| Piper spp. Extract | 1 | mg |

For each of the components there may be more than one source for the ingredient. Vitamin A palmitate and beta-carotene are sources of Vitamin A. For Vitamin C, ascorbic acid may be a preferred source of Vitamin C, but other forms of Vitamin C, including sodium ascorbate, can be used in lieu of or in combination with ascorbic acid. Cholecalciferol is a source of Vitamin D. D-alpha tocopheryl succinate and mixed tocopherols are sources of Vitamin E. Natural and mixed carotenoids are also sources of Vitamin E. For zinc, zinc oxide may be used and provides the most concentrated form of elemental zinc. Zinc gluconate and zinc chelate [monomethionine] are also sources of zinc. Copper oxide is a form of copper that is frequently used in dietary supplements, but alternative forms such as copper gluconate and copper amino acid chelate can also be used. The algae Haematococcus pluvialis, cultivated in Hawai'i, is a known starting material for producing an extract contining astaxanthin. Omega-3 fatty acids, including eicosapentaenoic acid (EPA) and docosahexaenoic acid (DHA) can derive from small feeder fish typically found at or near the bottom of the food chain, including sardines, anchovies, and mackerel. These marine species are advantageously devoid of the contaminants typically associated with more predatory, higher marine species.

Blending in suitable devices combines the components. For example, mixing can occur in a V-type blender. One of ordinary skill in the art can determine the devices and apparatuses best suited for combining the components of the mixture comprising non-essential natural antioxidants and chemoprevention agents.

### Administration of the Compositions to Promote Ocular Health

Embodiments provide methods of administering compositions to promote ocular health. Daily administration of the daily dose of the composition ameliorates stresses and degenerations. The compositions, which contain certain amounts of multivitamins, trace elements, non-essential antioxidants and fatty acids, are useful when administered daily for ameliorating oxidative and visual stresses and degradation of the eye due to age-related macular degeneration (AMD), diabetes, and hyperglycemia.

Embodiment methods include self-introduced administration, which makes oneself the subject of the daily administration. Examples of self-introduction include orally consuming the composition with meals or as capsules, injecting oneself with a solution comprising the composition, and applying an ointment comprising the composition to one's skin. Other embodiment methods include administering compositions to the subject that is not oneself. Examples include feeding the subject a foodstuff comprising the composition as part of a daily meal and injecting a subject with a solution comprising the composition. One of ordinary skill in the art can device numerous methods of administering compositions to promote ocular health to various subjects to effect the proper daily dose. These can include time-release capsules, orally ingested liquids, intraperitoneal, intravenous, subcutaneous, sublingual, transcutaneous, intramuscular, and other well-understood forms of administration of composition to promote ocular health.

“Subjects” include, without limitation, animals, which include mammals, which include dogs, cats, mice and humans (homo sapiens).

Compositions to promote ocular health are in “daily dose” amounts. That is, the compositions as described represent the amount of the composition for administration during a 24-hour period or on a daily basis to a subject to ameliorate oxidative and visual stresses and degradation of the eye due to age-related macular degeneration (AMD), diabetes, and hyperglycemia. Visual stress occurs from exposure to visual display terminals, including computer monitors, smart phones, laptops, and tablet personal computers.

In some embodiments the administration of the daily dose of the composition occurs on a continuing daily basis after the diagnosing the subject with the degradation of the eye. In such embodiments, the method includes a step for diagnosing the subject with a degradation of the eye, which can be due to age-related macular degeneration, diabetes, hyperglycemia, dry eye, and other illnesses and age-related conditions. In other embodiments, the administration of the daily dose of the composition occurs on a continuing daily basis after exposure to a source of oxidative or visual stress to the eye, including visual display terminals.

Some embodiments administer pure, singular or refined compositions to the subject. Typically, blending with other materials for ingestion or injection occurs. Dilution for making compositions for oral administration can use foodstuffs (water, drinks, meals, chow mixes) edible solids, gels; palatable liquids and solutions; inert binding materials; excipients, including soybean oil, white beeswax, and soy lethicin; and inert materials that are not harmful if consumed or in contact with mucus and ocular membranes of a mammalian body, especially a human being. Saline and other fluids known to those skilled in the art can be used for making intravenous administration compositions.

Oral consumption is the preferred embodiment of administration to the subject. The act of digestion by the subject metabolizes many of the components of the composition, especially antioxidant compounds, and converts them into their active and protective forms. Oral consumption is also a comfortable and palatable delivery vehicle for introduction of the compositions versus more invasive means given the intention of daily administration. Forms of the composition for oral administration, either in pure or diluted form, include lacquered or coated tablets, unlacquered or uncoated tablets, caplets, hard capsules, liquid-filled capsules, hard gelatin capsule, hard vegetable-based capsule, elixir, soft-chew, lozenge, chewable bar, juice suspension, liquids, time-release formulations, and foodstuffs. In the preferred form, the composition is contained in an easy-to-swallow, oblong soft gelatin capsule with an opaque caramel color that shields the active ingredients from degradation due to the intrusion of light.

If a footstuff or other material for oral consumption is used for embodiment administration, it is preferable that components of the foodstuff or other materials do not react with, interfere with the processing or absorption of, or negate the desirable properties of the composition to promote ocular health.

Embodiment administrations include using of one or more capsules containing at least a portion of the composition to promote ocular health. The formulation of an individual capsule is determined based on the amount of the essential ingredients that are required to be present in each capsule to total the amount of essential ingredients. For simplicity, during the remaining portion of this description, the form of administration, whether lacquered tablets, unlacquered tablets, caplets or capsules, will be referred to as “capsules” without distinguishing among the various forms.

Embodiment administrations of the daily dose can provide one capsule for the entirety of the daily dose administration or multiple capsules proportionated according to the number of administration during the day. The entire daily dose of the composition does not have to be administered in a single dose during a given 24-hour period. In some embodiment administrations the daily dose of the composition is sub-divided and proportionally administered more than once per day to provide the subject with the appropriate daily dose amount within a given day. The daily dose apportionment reflects the frequency of administrations necessary in a 24-hour cycle to achieve proper daily dosage of the composition. For example, it may be easier to administer the daily dose of composition as three, one-third portions three times a day. In this example, tri-daily consumption of one-third portions of the daily dose of composition can occur with three regularly scheduled meals or as three, one-third daily dose capsules and therefore effect the daily dose for the subject. Dividing the daily dose into smaller, more frequent administrations can improve the habit of self-administration, make it easier to audit for determining proper dosage of the subject during a 24-hour period, and make consumption of the composition more tolerable to those with highly-sensitive taste. The sum of the proportional amounts of the administered composition during the 24-hour period should total the daily dose of the composition to achieve the benefits of ameliorating oxidative and visual stresses and degradation of the eye, including age-related macular degeneration (AMD).

Research suggests that fat soluble antioxidants such as carotenoid lutein are best absorbed when combined with fat (e.g. oil). Advantageously, the composition contains molecularly distilled fish oil as a source of omega-3 fatty acids, which also acts as a carrier and solubilizer for these carotenoids. This reduces the need to take the composition with a fatty meal. Although not intending to be bound by theory, it is believed that combining a partial or entire daily dose with the intake of a small meal containing a healthy portion of fat (e.g., olive oil, salmon) may further help in the proper assimilation of the active components. It is preferable to avoid taking at the same time as foods rich in oxalic or phytic acid (e.g., raw beans, seeds, grains, soy, spinach, rhubarb) as they may depress the absorption of minerals like zinc; however, it is not necessary to avoid these foods for the composition to still be effective.

The actual capsules for consumer use may contain somewhat more than the total amounts specified as the daily dose. The active ingredients may degrade over time. Consequently, in order to assure that the active ingredients are presented in the minimum amounts required at the time by the subjects, formulating capsules comprising a composition to promote ocular health may require increasing the dosage present in the capsule beyond the minimum amount required in order to account for and compensate for degradation of the composition with time. Some of the essential ingredients degrade faster than others, which can result in different percentages of excess in each capsule for one essential ingredient as compared to a different essential ingredient.

### Pharmacology

Oxidative stress to the retina may be involved in the pathogenesis of several conditions leading to visual decline, both in normal as well as diseased individuals. Dietary antioxidants play a role in neutralizing free radicals caused by physiological factors such as excessive mitochondrial activity and hyperglycemia, as well as environmental factors such as exposure to ultraviolet light.

It is well documented that vitamin A deficiency can result in night blindness and blindness due to the erosion of the cornea, but recent evidence suggests that preformed vitamin A may positively impact vision in individuals who are not vitamin A-deficient, possibly by virtue of its antioxidant and immunomodulatory properties. Furthermore, vitamin A is known to modulate retinal pigment epithelial (RPE) cellular function and behavior by helping to restore visual pigment and function.

Vitamin C is arguably the most important water-soluble biological antioxidant. It can scavenge both reactive oxygen species (ROS) and reactive nitrogen species thought to play roles in tissue injury associated with the pathogenesis of various conditions. By virtue of this activity, it inhibits lipid peroxidation, oxidative DNA damage and oxidative protein damage. It helps preserve intracellular reduced glutathione concentrations, which in turn helps maintain nitric oxide levels and potentiates its vasoactive effects. In addition, vitamin C may modulate prostaglandin synthesis to favor the production of eicosanoids with antithrombotic and vasodilatory activity. Some studies suggest a protective effect against cataracts. Age-related lens opacities are thought to be due to oxidative stress. Ocular tissue concentrates vitamin C, and its antioxidant action could account for its possible effect in protection against visual decline.

Vitamin D has immunomodulatory activity. It is known that serum levels of vitamin D are inversely associated with age-related visual decline and early stages of macular structural damage. Though the pharmacodynamics are not fully understood, it is believed that vitamin D offers a protective effect against retinal oxidative damage. Furthermore, vitamin D acts as an inhibitor of retinal neovascularization in animal models.

The mechanisms underlying the immune effects of zinc are not fully understood, though some of them may be accounted for by its membrane-stabilization effect. Zinc is also believed to have secondary antioxidant activity. Although zinc does not have any direct redox activity under physiological conditions, it nevertheless may influence membrane structure by its ability to stabilize thiol groups and phospholipids. It may also occupy sites that might otherwise contain redox active metals such as iron. These effects may protect membranes against oxidative damage. Zinc also comprises the structure of copper/zinc superoxide dismutase (Cu/Zn SOD), a very powerful antioxidant. Additionally, it may have secondary antioxidant activity via the copper-binding protein metallothionein.

The carotenoids lutein and zeaxanthin are naturally present in the macula. They filter out potentially phototoxic blue light and near-ultraviolet radiation from the retina. The protective effect is due in part, to the reactive oxygen species (ROS) quenching ability of these carotenoids. Zeaxanthin is the predominant pigment in the fovea, the region at the center of the macula. The quantity of zeaxanthin gradually decreases and the quantity of lutein gradually increases in the region surrounding the fovea, and lutein is the predominant pigment at the outermost periphery of the macula. Lutein and zeaxanthin also are the only two carotenoids that have been identified in the human lens. They may offer some protection against age-related increases in lens density and possibly cataract formation.

Unlike lutein and zeaxanthin, astaxanthin, another xanthophyll carotenoid, is not a retinal pigment. Astaxanthin has both lipo- and hydrophilic antioxidant activity, working both inside as well as outside cell membranes. Astaxanthin is known to cross the blood-brain barrier and effectively work inside retinal tissues. Evidence suggests it inhibits the neurotoxicity induced by peroxide radicals or serum deprivation; reduces the intracellular oxidation induced by various reactive oxygen species (ROS); decreases the radical generation induced by serum deprivation in RGC-5 (retinal ganglion cells); and ameliorates the retinal damage (a decrease in retinal ganglion cells and in thickness of inner plexiform layer) induced by chemical and environmental factors. Furthermore, astaxanthin reduced the expressions of 4-hydroxy-2-nonenal (4-HNE)-modified protein (indicator of lipid peroxidation) and 8-hydroxy-deoxyguanosine (8-OHdG; indicator of oxidative DNA damage) in animal models. These findings indicate that astaxanthin has neuroprotective effects against retinal damage in-vivo, and that its protective effects may be partly mediated via its antioxidant effects. Moreover, astaxanthin has been shown to increase muscular fiber endurance through improved muscle lipid metabolism via inhibitory effect of oxidative CPT I (carnitine palmitoyl transferase—type 1) modification, which may account for documented improvements in eye strain and accommodation in visual display terminal workers, as well as visual acuity and endurance.

Eicosapentaenoic acid (EPA) and docosahexaenoic acid (DHA) are essential omega-3 fatty acids and both play a role in the formation of anti-inflammatory and immunemodulating eicosanoids. As such, they have several actions in a number of body systems. Both play an important role in the maintenance of normal blood flow as they lower fibrinogen levels. DHA is vital for normal neurological function throughout life. Several mechanisms are believed to account for the anti-inflammatory activity of EPA and DHA. Most notably, the two competitively inhibit the conversion of arachidonic acid to the pro-inflammatory prostaglandin E2 (PGE2), and leukotriene B4 (LKB4), thus reducing their synthesis. EPA and DHA also inhibit the synthesis of the inflammatory cytokines Tumor Necrosis Factor-alpha (TNF-α), Interleukin-1 (IL-1) beta. EPA and DHA inhibit the 5-LOX (lipoxygenase) pathway responsible for the conversion of arachidonic acid to inflammatory leukotrienes in neutrophils and monocytes and can suppress phospholipase C-mediated signal transduction, also involved in inflammatory events. EPA is the precursor to series-3 prostaglandins, series-5 leukotrienes (LTBS) and series-3 thromboxanes (TXA3). This could account in part for its microvascular and anti-inflammatory role. Furthermore, EPA is a precursor of resolvins (Rv) such as RvE1 and RvD1 which may help reduce tear gland inflammation, increase tear volume and ocular lubrication.

EPA and DHA have both similar and dissimilar physiologic roles. EPA appears to be more important in those roles where the eicosanoids are involved such as inflammation as well as tear gland function and tear production, whereas DHA seems to play its most important role in offering structural protection to the retina and other neurovascular structures such as corneal nerves.

Taurine has antioxidant activity derived from its ability to scavenge the reactive oxygen species (ROS) hypochlorite to form the relatively harmless N-chlorotaurine, which is then reduced to taurine and chloride. This activity may protect against collateral tissue damage that can occur from the respiratory burst of neutrophils in the retina. Taurine also appears to modulate the activation of cGMP gated channels, which control the influx of calcium into the rod outer segments, the function of which is critical in the phototransduction process. Taurine may also suppress peroxidation of membrane lipoproteins by other ROS. It is thought that this effect is not due to taurine's scavenging of these ROS, but rather to taurine's membrane-stabilizing activity, which confers greater resistance to the membrane lipoproteins against lipid peroxidation.

Alpha-lipoic acid (ALA) forms a redox couple with its metabolite, dihydrolipoic acid (DHLA) and may scavenge a wide range of reactive oxygen species. Both ALA and DHLA can scavenge hydroxyl radicals, nitric oxide radicals, peroxynitrite, hydrogen peroxide and hypochlorite. ALA, but not DHLA, may scavenge singlet oxygen, and DHLA, but not ALA, may scavenge superoxide and peroxyl reactive oxygen species.

ALA has been found to decrease urinary isoprostanes, O-LDL and plasma protein carbonyls, markers of oxidative stress. Furthermore, ALA and DHLA have been found to have antioxidant activity in aqueous as well as lipophilic regions, and in both extracellular as well as intracellular environments. ALA is also involved in the recycling of other biological antioxidants such as vitamins C and E, as well as glutathione. Finally, preliminary scientific evidence suggests a protective effect in the retina against ischemia and elevated blood sugar levels, such as is commonly seen in diabetic patients.

Pine bark bioflavonoids have demonstrated a number of antioxidant and vasoprotective activities, including scavenging of the superoxide radical anion, hydroxyl radical, lipid peroxyl radical, peroxynitrite radical, and singlet oxygen. Pharmacological studies employing in vitro, animal, and human models have found that pine bark and its bioflavonoids have potent anti-inflammatory actions, improve endothelial function (produce vasodilatation), reduce platelet aggregation, reduce alpha-glucosidase activity and blood glucose levels, and promote wound healing through mechanisms not yet fully understood. They have also been shown to protect low-density lipoprotein (LDL) from oxidation. It has been suggested that pine bark flavonoids may bind to the blood vessel wall proteins and mucopolysaccharides, and produce a capillary sealing effect, leading to a reduced permeability and edema formation, which may account for their protective effect in the eye.

Piperine, a chemical constituent of the black pepper (Piper spp.) has bioavailabity enhancing activity of certain nutrients, including antioxidants of the carotenoid family (i.e. lutein, zeaxanthin, etc) as well as several vitamins and minerals. The mechanism of action is not completely understood, but experiments done both in-vitro and in-vivo suggest that it may operate by increasing either membrane fluidity and affinity of nutrients to the cell membrane, or solubilization of the intracellular lipid moiety in the epithelial gastrointestinal tissues due to its lipophilic nature, making it more permeable to the applied nutrient.

### Pharmacokinetics

Vitamin A (retinyl palmitate ester) is hydrolyzed by a pancreatic hydrolase and combined with bile acids and other fats prior to its uptake by enterocytes in the form of micelles. It is then re-esterified and secreted by the enterocytes into the lymphatic system in the form of chylomicrons. These chylomicrons enter the circulation via the thoracic duct and undergo metabolism via lipoprotein lipase. Most of the retinyl esters are then rapidly taken up into liver parenchymal cells and again hydrolyzed to all-trans retinol and fatty acids (e.g. palmitate). All-trans retinol may be then stored by the liver as retinyl esters or transported in the circulation bound to serum retinol binding protein (RBP). Serum RBP is the principal carrier of retinol, which comprises greater than 90% of serum vitamin A. It is believed that RBP in association with transthyretin or prealbumin co-transport proteins are responsible for the transport of retinol into target cells. All-trans retinol is delivered to the cornea via the tears and by diffusion through eye tissue. Retinol is oxidized to retinal via retinol dehydrogenase. Retinal is metabolized to retinoic acid via retinal dehydrogenase. The metabolites of retinol and retinoic acid undergo gucuronidation, glucosylation and amino acylation. They are excreted mainly via the biliary route, though some excretion of retinol and its metabolites also occurs via the kidneys.

Intestinal absorption of vitamin C occurs primarily via a sodium-dependent active transport process, although some diffusion may also come into play. The major intestinal transporter is SVCT1 (sodium-dependent vitamin C transporter 1). Some ascorbic acid may be oxidized to dehydroascorbic (DHAA) acid and transported into enterocytes via glucose transporters. Within the enterocytes, all DHAA is reduced to ascorbic acid via reduced glutathione, and ascorbic acid leaves the enterocytes to enter the portal and systemic circulation for distribution throughout the body. The transporter SVCT2 appears to aid in the transport of vitamin C into the aqueous humor of the eyes. Though it cannot itself cross the blood-brain barrier, ascorbic acid may be oxidized to DHAA and be transported to the brain tissues via GLUT1 (glucose transporter 1), where it can then be reduced back to ascorbic acid for utilization. Metabolism and excretion of vitamin C occurs primarily via oxidation to DHAA and hydrolyzation to diketogulonate, though other metabolites such as oxalic acid, threonic acid, L-xylose and ascorbate-2-sulfate can also result. The principal route of excretion is via the kidneys.

Vitamin D is principally absorbed in the small intestine via passive diffusion. It is delivered to the enterocytes in micelles formed from bile acids, fats, and other substances. Like vitamin A, vitamin D is secreted by the enterocytes into the lymphatic system in the form of chylomicrons and enters the circulation via the thoracic duct. It is also transported in the blood bound to an alpha globulin known as Vitamin D-Binding Protein (DBP) and the group-specific component (Gc) protein. Much of the circulating vitamin D is extracted by the hepatocytes to be metabolized to 25-hydroxyvitamin D [25(OH)D] or calcidiol via the enzyme vitamin D 25-hydroxylase. 25(OH)D is then metabolized in the kidney to the biologically active hormone form of vitamin D, calcitrol [1,25(OH)2D], via the enzyme 25-hydroxyvitamin D-1-alpha-hydroxylase. Calcitrol may undergo further hydroxylation and metabolism into 24,25(OH)2D and 1,24,25(OH)3D. These metabolites, as well as vitamin D are excreted primarily via the biliary route. The final degradation product of 1,25(OH)2D is calcitroic acid, which is excreted by the kidney.

Much of the pharmacokinetics of zinc in humans remains unknown. Zinc is absorbed all along the small intestine, though most appears to be assimilated from the jejunum. Zinc uptake across the brush border appears to occur by both a saturable barrier-mediated mechanism and a non-saturable non-mediated mechanism. The exact mechanism of zinc amino-acid chelates (such as the zinc-methionine used in AmeriSciences OS2) transport into the enterocytes remains unclear, but evidence demonstrates greater bioavailability than other supplemental forms. Zinc transporters have been identified in animal models. Once the mineral is within the enterocytes, it can be used for zinc-dependent processes, become bound to metallothionein and held within the enterocytes or pass through the cell. Transport of zinc across the serosal membrane is carier-mediated and energy-dependent. Zinc is transported to the liver via the portal circulation. A fraction of zinc is extracted by the hepatocytes, and the remaining zinc is transported to the various cells of the body via the systemic circulation. It is transported bound to albumin (about 80%), alpha-3-macroglobulin (about 18%), and to such proteins as transferin and ceruloplasmin. The major route of zinc excretion appears to be the gastrointestinal tract via biliary, pancreatic or other gastrointestinal secretions. Fecal zinc is also comprised of unabsorbed dietary zinc as well as the sloughing of mucosal cells.

Carotenoids such as lutein and zeaxanthin appear to be more efficiently absorbed when administered with high-fat meals. They are hydrolyzed in the small intestine via esterates and lipases, and solubilized in the lipid core of micelles formed from bile acids and other lipids. They can also form clathrate complexes with conjugated bile salts. Both of these complexes can deliver carotenoids to the enterocytes, where they are then released into the lymphatics in the form of chylomicrons. From there, they are transported to the general circulation via the thoracic duct. Lipoprotein lipases hydrolyze much of the triglyceride content in the chylomicrons found in the circulation, resulting in the formation of chylomicrons remnants, which in turn retain apolipoproteins E and B48 on their surfaces and are mainly taken up by the hepatocytes. Within the liver, carotenoids are incorporated into lipoproteins and they appear to be released into the blood mainly in the form of HDL and—to a much lesser extent—VLDL. Lutein and zeaxanthin are mainly accumulated in the macula of the retina, where they bind to the retinal protein tuberlin. Zeaxanthin is specifically concentrated in the fovea. Lutein is distributed throughout the retina. Astaxanthin, on the other hand, is distributed throughout the body, with muscle tissue seemingly receiving larger concentrations based on tissue/plasma ratio at 8 and 24 hours after oral ingestion. Lutein appears to undergo some metabolism in-situ to meso-zeaxathin. Xanthophylls as well as their metabolites are believed to be excreted via the bile and, to a lesser extent, the kidney.

Following ingestion, EPA and DHA undergo hydrolysis via lipases to form monoglycerides and free fatty acids. In the enterocytes, reacylation takes place and results in the formation of triacylglycerols, which are assembled with phospholipids, cholesterol and apoproteins into chylomicrons. These are released into the lymphatic system from whence they are transported to the systemic circulation. Here, the chylomicrons are degraded by lipoprotein lipase, and EPA & DHA are transported to various tissues of the body via blood vessels, where they are used mainly for the synthesis of phospholipids. Phospholipids are incorporated into the cell membranes of red blood cells, platelets, neurons and others. EPA and DHA are mainly found in the phospholipid components of cell membranes. DHA is taken up by the brain and retina in preference to other fatty acids. DHA can be partially and conditionally re-converted into EPA, and vice-versa, although the process is thought to be less-than-efficient and may be adversely affected by age.

Although not an amino-acid in the true sense of the word, taurine is absorbed from the small intestine via the beta-amino acid transport system: a carrier system dependent on sodium and chloride that serves gamma-aminobutyric acid and beta-alanine, as well as taurine. It is transported to the liver via the portal circulation, where much of it forms conjugates with bile acids. Taurocholate (the bile salt conjugate of taurine and cholic acid) is the principal conjugate formed via the enzyme choloyl-CoA N-acyltransferase. Taurine conjugates are excreted through the bile. Remaining taurine that is not conjugated or used in the biliary process is distributed via the systemic circulation to various tissues in the body, including the retina and other eye tissues. Taurine is not usually completely reabsorbed from the kidneys, and fractions of ingested taurine are excreted in the urine.

Alpha lipoic acid pharmacokinetic data demonstrate that its absorption takes place from the small intestine, followed by portal circulation delivery to the liver, and to various tissues in the body via systemic circulation. Alpha lipoic acid readily crosses the bloodbrain barrier, and is readily found (following distribution to the various tissues) extracellularly, intracellularly and intramitochondrially. It is metabolized to its reduced form, dihydrolipoic acid (DHLA) by mitochondrial lipoamide dehydrogenase, which can in turn form a redox couple with lipoic acid. ALA is also metabolized to lipoamide, which forms an important cofactor in the multienzyme complexes that catalyze pyruvate and alpha-ketoglutarate, both important aspects of cellular respiration and energy production via the Krebs cycle. ALA can also be metabolized to dithiol octanoic acid, which can undergo catabolism.

The pharmacokinetics of bioflavonoids such as those found in pine bark and piperine found in Piper species are not fully understood in humans. It is known, however, that pine bark flavonoids undergo extensive glucuronidation and sulfation during and following absorption from the small intestine. Both glucoronides and sulfates, as well as other metabolites are primarily excreted through the urine. In animals, piperine is absorbed following ingestion, and some metabolites have been identified, such as piperonylic acid, piperonyl alcohol, piperonal and vanillic acid are found in the urine. One metabolite, piperic acid, is found in the bile. Most publications conclude that further pharmacokinetic studies are needed to fully understand if this data is applicable to humans as well.

Where a range of values is provided in the Specification or in the appended Claims, it is understood that each intervening value between the upper limit and the lower limit within the provided range as well as the upper limit and the lower limit are encompassed in the invention. It is also understood that that any one or more of the intervening or limit values can act as a limit or limits for a smaller range of values, which is encompassed in the invention, within the range of provided values. The smaller range of values is encompassed by the invention subject to any specific exclusion of a portion of the provided range of values.

Unless defined otherwise, all technical and scientific terms used in the Specification and appended Claims have the same meaning as commonly understood by one of ordinary skill in the art to which this invention belongs. Although any methods and materials similar or equivalent to those described can also be used in the practice or testing of the invention, a limited number of the exemplary methods and materials are described.

As used in the Specification and appended Claims, the singular forms “a”, “an”, and “the” include plural references unless the context clearly indicates otherwise.

It should be apparent to those skilled in the art that many more modifications besides those already described are possible without departing from the inventive concepts. The inventive subject matter, therefore, is not restricted except in the spirit of the disclosure.

In interpreting the Specification and appended Claims, all terms should be interpreted in the broadest possible manner consistent with the context of each term. In particular, the terms “comprises” and “comprising” should be interpreted as referring to elements, components or steps in a non-exclusive manner, indicating that the referenced elements, components or steps may be present, or utilized, or combined with other elements, components, or steps that are not expressly referenced.

Where reference is made in the Specification and appended Claims to a method comprising two or more defined steps, the defined steps can be carried out in any order or simultaneously (except where the context excludes that possibility) The method can include one or more other steps which are carried out before any of the defined steps, between two of the defined steps, or after all the defined steps (except where the context excludes that possibility).

What is claimed is:

1. A composition to promote ocular health for ameliorating oxidative and visual stresses and degradation of the eye, including due to age-related macular degeneration (AMD), the composition to promote ocular health comprising:
an amount of vitamin A in a range of from about 2500 to about 50000 IU, where the vitamin A further comprises beta-carotene in a range of from about 0 to about 50000 IU;
an amount of vitamin C in a range of from about 60 to about 500 mg;
an amount of vitamin D in a range of from about 400 to about 2000 IU;
an amount of vitamin E in a range of from about 0 to about 400 IU;
an amount of zinc in a range of from about 15 to about 80 mg;
an amount of copper in a range of from about 0 to about 2 mg;
an amount of selenium in a range of from about 35 to about 200 μg;
an amount of non-vitamin A carotenoids in a range of from about 5 to about 62 mg, where the non-vitamin A carotenoids further comprises lutein in a range of from about 5 to about 50 mg and zeaxanthin in a range of from about 0.25 to about 12 mg;
an amount of omega-3 fatty acids in a range of from about 0 to about 5000 mg, where the omega-3 fatty acids further comprises eicosapentaenoic acid in a range of from about 0 to about 5000 mg and docosahexaenoic acid in a range of from about 0 to about 3000 mg;
an amount of taurine in a range of from about 100 to about 1000 mg;
an amount of alpha lipoic acid in a range of from about 0 to about 1000 mg;
an amount of pine bark extract in a range of from about 0 to about 500 mg;
an amount of astaxanthin in a range of from about 0 to about 5 mg;
an amount of Piper spp. extract in a range of from about 0 to about 5 mg.

2. The composition of claim 1 where there is no amount of copper present in the composition and the amount of zinc in the composition is less than or about 30 mg.

3. The composition of claim 1 where the amount of copper in the composition is in a range of from about 1 to about 2 mg and the amount of zinc in the composition is in a range of from about 30 mg to about 80 mg.

4. The composition of claim 1 where there is no amount of beta-carotene present in the composition.

5. The composition of claim 1 where there is no amount of vitamin E present in the composition.

6. The composition of claim 1 where the amount of total non-vitamin A carotenoids is in a range of from about 0 to about 12 mg.

7. The composition of claim 1 comprising:
vitamin A in an amount of about 2500 IU;
vitamin C in an amount of about 250 mg;
vitamin D in an amount of about 800 IU;
zinc in an amount of about 30 mg;
selenium in an amount of about 70 μg;
non-vitamin A carotenoids in an amount of about 12 mg, where the non-vitamin A carotenoids further comprises lutein in an amount of about 10 mg and zeaxanthin in an amount of about 2 mg;
omega-3 fatty acids in an amount of about 500 mg, where the omega-3 fatty acids further comprises eicosapentaenoic acid in an amount of about 300 mg and docosahexaenoic acid in an amount of about 200 mg;
taurine in an amount of about 500 mg;
alpha lipoic acid in an amount of about 100 mg;
pine bark extract in an amount of about 10 mg;
astaxanthin in an amount of about 1 mg; and
Piper spp. extract in an amount of about 1 mg.

8. The composition of claim 7 where there is no amount of beta-carotene, vitamin E and copper present in the composition.

9. A method of treatment for a subject exposed to a source of oxidative or visual stress to the eye or having a degradation of the eye, the method of treatment comprising the steps of:
administering to the subject a daily dose of the composition to promote ocular health of claim 1
such that the effects induced by the oxidative or visual stress source or the degradation of the eye are ameliorated.

10. The method of claim 9 where the administration of the daily dose of the composition to promote ocular health occurs on a continuing daily basis after exposure to the source of oxidative or visual stress to the eye.

11. The method of claim 10 where the source of visual stress is a visual display terminal.

12. The method of claim 9 further comprising the step of diagnosing the subject with the degradation of the eye.

13. The method of claim 11 where the administration of the daily dose of the composition to promote ocular health occurs on a continuing daily basis after the diagnosing the subject with the degradation of the eye.

14. The method of claim 11 where the degradation of the eye is due to age-related macular degeneration (AMD).

15. The method of claim 11 where the degradation of the eye is due to diabetes.

16. The method of claim 11 where the degradation of the eye is due to hyperglycemia.

17. The method of claim 9 where the subject is a human being.

18. The method of claim 9 where the daily dose of the composition to promote ocular health is administered proportionally during a 24-hour period such that the sum of the proportional amounts of the administered composition to promote ocular health during the 24-hour period totals the daily dose.

19. The method of claim 9 where the daily dose of the composition to promote ocular health is administered orally as part of a composition for oral administration, the oral administration composition selected from the group comprising lacquered tables, coated tablets, unlacquered tablets, uncoated tablets, caplets, hard capsules, liquid-filled capsules, hard gelatin capsules, hard vegetable-based capsules, elixirs, soft-chews, lozenges, chewable bars, juice suspensions, liquids, time-release formulations, and foodstuffs.

20. The method of claim 19 where the composition for oral administration further comprises an excipient selected from the group comprising soybean oil, white beeswax, soy lethicin, and combinations thereof.
